(19) United States
(12) Patent Application Publication
(10) Pub. No.: US 2008/0113444 A1
(43) Pub. Date: May 15, 2008

(54) Method for detecting oligermization of soluble amyloid beta oligomers

(76) Inventor: Todd R. Pray, San Mateo, CA (US)

Correspondence Address: Jane Massey Licata;Licata & Tyrrell P.C., 66 E. Main Street, Marlton, NJ, 08053, US

(21) Appl. No.: 11/872,775

(22) Filed: Oct. 16, 2007

Related U.S. Application Data
(60) Provisional application No. 60/829,824, filed on Oct. 17, 2006.

(51) Int. Cl. G01N 33/00

(52) U.S. Cl. 436/87

(57) ABSTRACT

The present invention relates to the assay, analysis, and characterization of soluble amyloid beta oligomers, as well as the characterization of inhibitors of soluble amyloid beta oligomer assembly. In particular, the present invention is a method for detecting assembly of soluble amyloid beta oligomers via a combination of fluorescence resonance energy transfer (FRET) or time-resolved FRET and fluorescence polarization (FP).

This application claims benefit of priority to U.S. Provisional Patent Application Ser. No. 60/829,824, filed Oct. 17, 2006, the content of which is incorporated herein by reference in its entirety.

### BACKGROUND OF THE INVENTION

Alzheimer's disease (AD) is a progressive and degenerative dementia (Terry, et al. (1991) Ann. Neurol. 30(4):572-80; Coyle (1987) in Encyclopedia of Neuroscience, Ed. G. Adelman, pp. 29-31, Birkhäuser: Boston-Basel-Stuttgart), which in its early stages manifests primarily as a profound inability to form new memories (Selkoe (2002) Science 298(5594):789-91). The amyloid β (Aβ) peptide was shown to be the major protein constituent of amyloid plaques and cerebrovascular amyloid deposits. Aβ 1-42 is quite hydrophobic and rapidly assembles into fibrils. Although it only represents 10-15% of the total Aβ peptide production, it is the predominant peptide in plaques, accompanied by smaller quantities of Aβ 1-43 and N-terminal truncated analogs of Aβ 1-42 and Aβ 1-43 (e.g., Aβ x-42 and Aβ x-43). Relatively little Aβ 1-40 deposits in plaques, due to its higher solubility, but it does assemble into fibrils at micromolar to millimolar concentrations in vitro. Fibrils prepared from synthetic Aβ 1-40 or Aβ 1-42 exhibit morphologies and Congo red birefringence similar to AD fibril deposits and both peptides can be toxic to neurons in culture. A number of studies demonstrated that Aβ neurotoxicity required prior assembly into fibrils (Lorenzo & Yankner (1994) Proc. Natl. Acad. Sci. USA 91(25):12243-7) and several reports have described trophic or cognition enhancing properties of the Aβ 1-40 at nanomolar concentrations. The link between fibrils and in vitro neurotoxicity was sufficient to convince many AD researchers that amyloid plaques were the cause of AD.

Despite these experimental results, a growing number of clinical and pathology studies suggested that plaques and fibrils were not responsible for cognitive deficits in AD. For example, careful analysis of plaque number and location revealed little or no correlation with nerve cell loss and cognitive impairment (Terry, et al. (1991) Ann. Neurol. 30(4):572-80; Terry, et al. (1999) “Alzheimer Disease”, 2^{nd} Edition, Lippincott Williams & Wilkins: Philadelphia, Pa.; McLean, et al. (1999) Ann. Neurol. 46(6):860-6; Hibbard & McKeel, Jr. (1997) Anal. Quant. Cytol. Histol. 19(2):123-38; Sze, et al. (1997) J. Neuropathol. Exp. Neurol. 56(8):933-44), and analysis of total amyloid load showed little correlation with disease severity (Giannakopoulos, et al. (2003) Neurology 60(9):1495-500). As transgenic mouse models capable of substantial Aβ 1-42 overproduction emerged, it became clear that significant behavioral deficits developed in these mice long before Aβ deposits or plaque pathology appeared. The parameter that correlated best with behavioral deficits was synaptic deterioration, a process with no apparent link to plaques or Aβ deposition (Mucke, et al. (2000) J. Neurosci. 20(11):4050-8; Hsia, et al. (1999) Proc. Natl. Acad. Sci. USA 96(6):3228-33; Kawarabayashi, et al. (2001) J. Neurosci. 21(2):372-81; Ashe (2005) Biochem. Soc. Trans. 33(Pt.4):591-4).

Passive immunization of transgenic hAPP mice cast further doubt on the role of amyloid plaques and fibrils (Dodart, et al. (2002) Nat. Neurosci. 5(5):452-7; Kotilinek, et al. (2002) J. Neurosci. 22(15):6331-5). The transgenic mice treated in these studies represented good models of early AD, as they developed age-dependent amyloid plaques and age-dependent memory dysfunction. When these mice were treated with monoclonal antibodies against Aβ, two surprising findings emerged, vaccinated mice showed reversal of memory loss within 24 hours of antibody injection, and improved cognitive function occurred with no change in amyloid plaque levels. These findings were completely at odds with a mechanism for memory loss that involved amyloid fibril-induced neuron death.

The disconnection between amyloid fibrils and neurotoxicity was established convincingly with the isolation, characterization, and analysis of amyloid-β derived diffusible ligands, ADDLs, (U.S. Pat. No. 6,218,506; Lambert, et al. (1998) Proc. Natl. Acad. Sci. USA 95(11):6448-53), which are neurotoxic soluble oligomeric assemblies of Aβ 1-42 with globular morphology distinct from deposited forms of Aβ.

ADDLs assemble from relatively low concentrations of Aβ 1-42, and they block LTP in intact animals or in hippocampal slice cultures (Lambert, et al. (1998) Proc. Natl. Acad. Sci. USA 95(11):6448-53; Wang, et al. (2002) Brain Res. 924(2):133-40; Wang, et al. (2004) J. Neurosci. 24(13):3370-8). ADDLs exert their memory-compromising activity, at least in part, by binding specifically to dendritic spines on hippocampal neurons (Lacor, et al. (2004) J. Neurosci. 24(45):10191-200) and they elevate phosphorylation of tau at AD-specific epitopes (Shughrue, et al. (2005) 2005 Abstract Viewer/Itinerary Planner Program No. 209.16 Washington, D.C.: Society for Neuroscience). ADDLs are substantially elevated in AD brain (Gong, et al. (2003) Proc. Natl. Acad. Sci. USA 100(18):10417-22) and in cerebrospinal fluid from AD-diagnosed individuals (Georganopoulou, et al. (2005) Proc. Natl. Acad. Sci. USA 102(7):2273-6), providing strong evidence that ADDLs are the relevant molecular pathogens in AD.

To facilitate the study of Aβ, various fluorescent-based methodologies have been developed. See, e.g., U.S. Pat. No. 6,927,401; U.S. Pat. No. 6,906,104; U.S. Pat. No. 6,905,827; U.S. Pat. No. 6,881,546; U.S. Pat. No. 6,864,290; U.S. Pat. No. 6,864,103; U.S. Pat. No. 6,858,383; U.S. Pat. No. 6,846,813; U.S. Pat. No. 6,828,106; U.S. Pat. No. 6,803,188; U.S. Pat. No. 6,770,448; U.S. Pat. No. 6,713,276; U.S. Pat. No. 6,600,017; U.S. Pat. No. 6,515,113; U.S. Pat. No. 6,495,664; U.S. Pat. No. 6,323,039; U.S. Pat. No. 6,294,330; U.S. Pat. No. 6,280,981; U.S. Pat. No. 6,197,928; U.S. Pat. No. 5,981,200; Kim & Lee (2004) Biochem. Biophys. Res. Commun. 316:393-397; Bacskai, et al. (2003) J. Biomed. Opt. 8:368-375; Gorman, et al. (2003) J. Mol. Biol. 325:743-757; Garzon-Rodrequez, et al. (1997) J. Biol. Chem. 272:21037-21044; Lindgren, et al. (2005) Biophys. J. 88:4200-4212; Lewis, et al. (2004) Neurobiol. Aging 25:1175-1185; Leissring, et al. (2003) J. Biol. Chem. 278:37314-37320; Taylor, et al. (2003) J. Protein Chem. 22:31-40; Allsop, et al. (2001) Biochem. Soc. Symp. 67:1-14; Allsop, et al. (2001) Biochem. Biophys. Res. Commun. 285:58-63; Huang, et al. (2000) J. Biol. Chem. 275:36436-36440.

The ADDL hypothesis provides a straightforward explanation for the early, subtle cognitive deficits in AD wherein low concentrations of ADDLs trigger abnormal neuronal signaling, and for the severe deficits in later-stage AD, wherein long-term exposure to increasing ADDL concentrations leads to progressive, degenerative pathology (e.g., neurofibrillary tangles) and neuron death. Given these considerations, soluble amyloid beta oligomers (including ADDLs) provide an optimum target for prophylactic and/or therapeutic treatment of Alzheimer's disease, Down's syndrome, mild cognitive impairment, and the like. The present invention addresses the need to assay, analyze, and characterize soluble amyloid beta oligomers (including ADDLs), including the need to identify, assay, analyze, and characterize inhibitors of the assembly and/or activity of these oligomers.

### SUMMARY OF THE INVENTION

The present invention is a method for detecting oligomerization of soluble amyloid beta peptides. The method of the present invention involves contacting a FRET-donor labeled amyloid beta subunit with a FRET-acceptor labeled amyloid beta subunit and measuring fluorescence resonance energy transfer (FRET) and fluorescence polarization (FP) of the subunits. In one embodiment, the FRET is time-resolved FRET (TR-FRET). In other embodiments, the method is carried out in the presence of one or more inhibitors to identify, assay, analyze, and characterize such inhibitors.

### BRIEF DESCRIPTION OF THE DRAWINGS

FIG. 1 depicts an assay of soluble amyloid beta oligomer (e.g., including, but not limited to, ADDLs, and the like) assembly using FRET. FRET occurs by proximity and overlap of donor emission and acceptor excitation dipoles.

FIG. 2 depicts results of an assay of selective Aβ(1-42) assembly using FRET showing relevant soluble amyloid beta oligomer species (FIG. 2A) versus a negative control (FIG. 2B) being formed during the reaction. These results were verified by atomic force microscopy (AFM), size exclusion chromatography (SEC), and neuronal binding.

FIG. 3 depicts the detection of ADDL assembly via FRET. FIG. 3A shows Aβ peptide antibody inhibition of ADDL assembly. Concentrations of antibody are indicated. FIG. 3B shows small molecule inhibition of ADDL assembly. Small molecule concentrations are indicated.

FIG. 4 depicts the detection of ADDL assembly using TR-FRET. FIG. 4A shows Aβ peptide antibody inhibition of ADDL assembly. Concentrations of antibody are indicated.

FIG. 4B shows small molecule inhibition of ADDL assembly. Small molecule concentrations are indicated.

FIG. 5 depicts the detection of ADDL assembly via FP and antibody-induced ADDL assembly inhibition, as well as the detection of antibody binding to ADDLs by FP.

FIG. 6 depicts results of optimizations of a soluble amyloid beta oligomer assembly assay using FRET and FP. The assays are typically performed at various conditions, including, but not limited to, in 384-well plates, at 37° C., and with (FIGS. 6B and 6D) or without (FIGS. 6A and 6C) a given stabilite (i.e., a composition of matter that stabilizes soluble amyloid beta oligomers, wherein such oligomers include, but not are not limited to, ADDLs, and the like). In the presence of a given stabilite there is a larger FP window (FIG. 6B) and tighter kinetic overlay and faster assembly (FIG. 6D).

FIG. 7 depicts exemplary high throughput screening (HTS) performance parameters for a soluble amyloid beta oligomer (e.g., including, but not limited to, ADDLs, and the like) assembly assay using FRET in the presence (+) and absence (−) of the stabilite, 100 mM MgCl₂. Z′≧0.7 were regularly observed in these assays. Z′≡1-3*(σ₁+σ₂)/(x₁+x₂).

FIG. 8 depicts exemplary high-throughput screening assay results for small molecule inhibitors (Compounds 1 and 2, FIGS. 8A and 8B; and Compounds 3 and 4, FIGS. 8C and 8D) of soluble amyloid beta oligomer assembly using FRET in the presence of the stabilite, 100 mM MgCl₂.

FIG. 9 depicts antibody-induced inhibition of soluble amyloid beta oligomer (e.g., including, but not limited to, ADDLs, and the like) assembly using assays disclosed and claimed herein. Similar potent assembly inhibition is seen in the presence of an anti-ADDL monoclonal antibody (FIG. 9A) and an anti-monomer polyclonal antibody (FIG. 9C), while very potent assembly inhibition is seen for at 300 nM for an anti-oligomer L polyclonal (“a”) (FIG. 9B).

FIG. 10 illustrates an exemplary fluorescence polarization (FP) assay according to embodiments of the present invention pertaining to carrying out FP in the presence of an antibody.

FIG. 11 depicts different FP profiles of soluble amyloid beta oligomer (e.g., including, but not limited to, ADDLs, and the like) assembly and binding by antibody reagents. FP elevation is observed at early time points (FIG. 11A and FIG. 11C), at later time points and intermediated concentrations (FIG. 11A) and intermediate FP values through the entire time course in inhibited samples (FIG. 11B).

FIG. 12 depicts results using FP assays for two different antibodies, mAb A (FIG. 12A) and mAb B (FIG. 12B), according to the embodiments disclosed herein showing different profiles of antibody binding to soluble amyloid beta peptide or oligomer (e.g., including, but not limited to, ADDLs, and the like) as well as ADDL inhibition. FP profiles such as these are used to rank polyclonal and monoclonal antibody response to different antigens.

FIG. 13 depicts antibody response ranking by ADDL inhibition using FRET kinetic profile assays according to the embodiments disclosed and claimed herein.

### DETAILED DESCRIPTION OF THE INVENTION

It has now been demonstrated that fluorescence resonance energy transfer (FRET) in combination with fluorescence polarization (FP) provides an effective means to assay, analyze, and characterize assembly of soluble amyloid beta oligomers containing Aβ, in particular Aβ (1-42) or Aβ (1-43), as well as truncations, analogs or mixtures thereof. Moreover, this combination of techniques is uniquely suitable for monitoring oligomerization in the presence of inhibitors such as small molecules, as well as in the presence of antibodies.

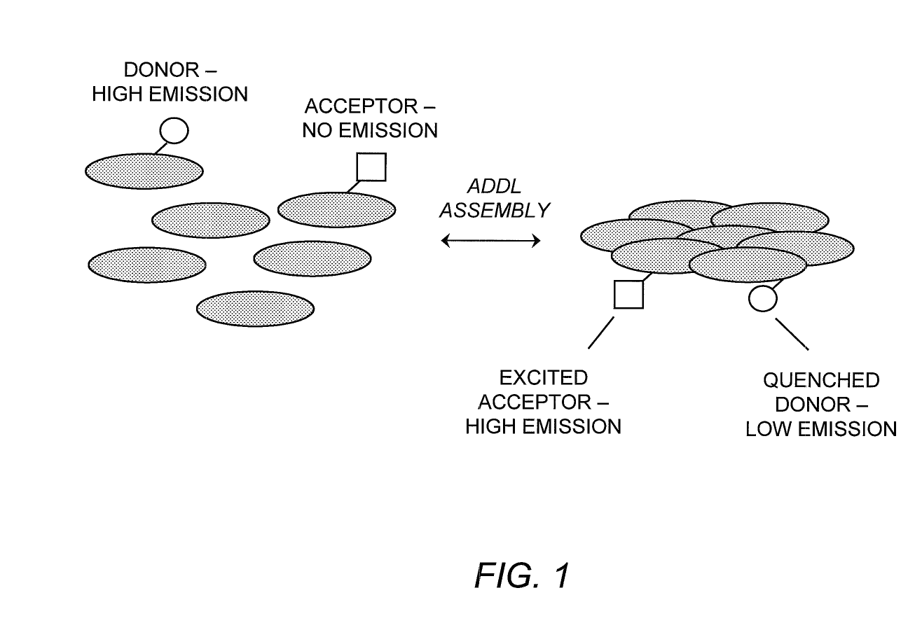

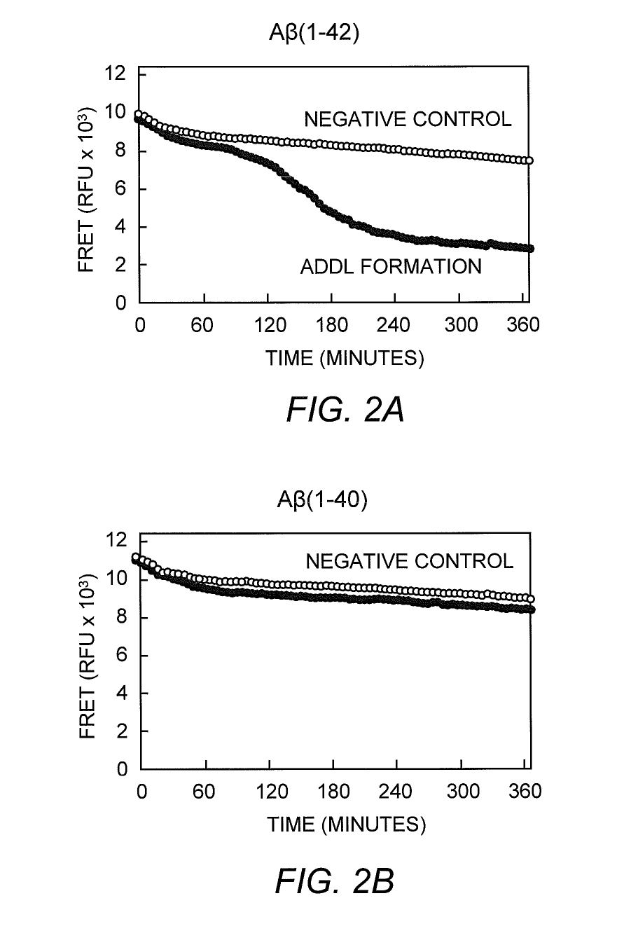

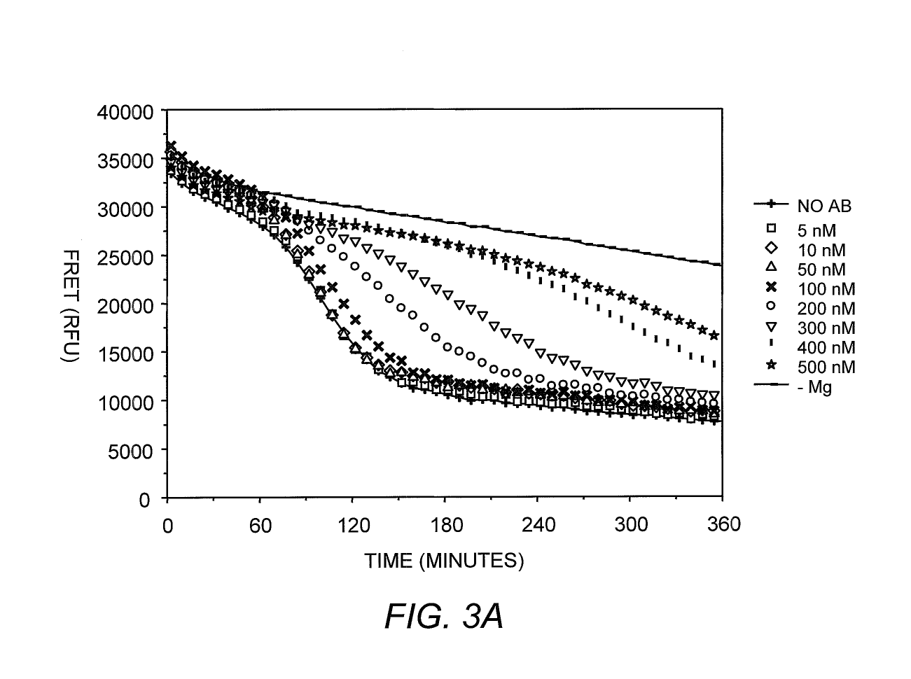

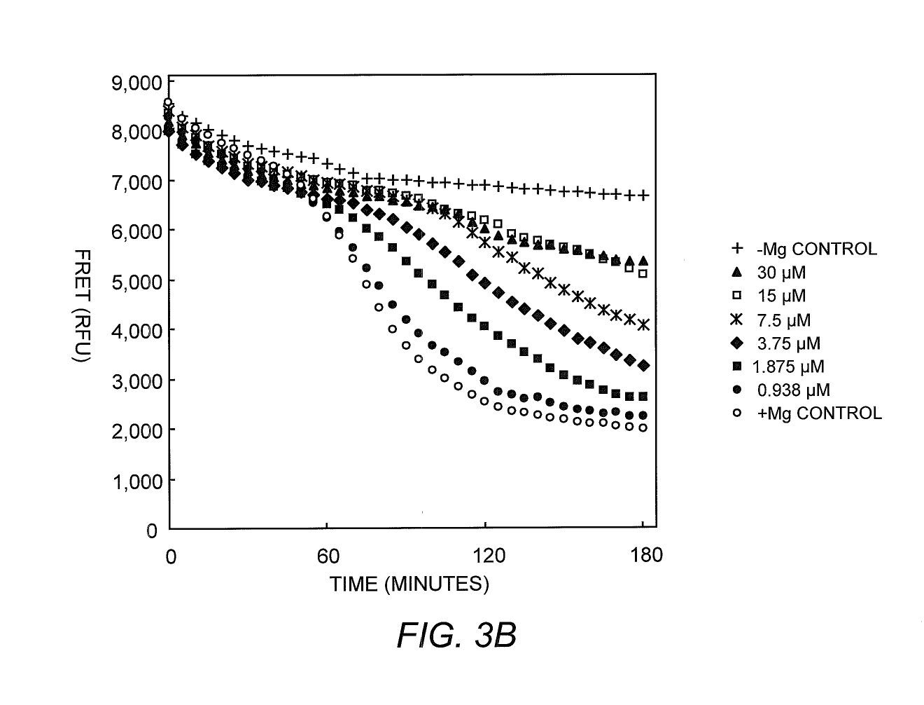

FRET occurs when a donor chromophore in its excited state transfers energy by a non-radiative long-range dipole-dipole coupling mechanism to an acceptor chromophore in close proximity (typically <10 nm). As a result, the acceptor emission is predominantly observed because of the intermolecular FRET from the donor to the acceptor (FIG. 1). FRET can be quantified in cuvette-based experiments or in microscopy images on a pixel-by-pixel basis. This quantification can be based directly (sensitized emission method) on detecting two emission channels under two different excitation conditions (primarily donor and primarily acceptor). However, for robustness reasons, FRET quantification is most often based on measuring changes in fluorescence intensity. An example of FRET analysis for detecting Aβ(1-42) assembly or oligomerization is shown in FIG. 2. An example of FRET analysis for detecting Aβ(1-42) assembly or oligomerization in the presence of an antibody or small molecule inhibitor is shown in FIGS. 3A and 3B, respectively.

It is contemplated that any suitable FRET donor-acceptor pair can be employed in accordance with the present invention. For example, cyan fluorescent protein (CFP) and yellow fluorescent protein (YFP) pair are known and routinely used as FRET pairs. Indeed, FRET assays are carried out in the art to measure, detect, identify, assay, analyze, and characterize various interactions and processes in biological systems. See e.g., Mitra, et al. (1996) Gene 173:13-17; De Angelis (1999) Physiol. Genomics 21:93-99; Latif & Graves (2000) Thyroid 10:407-412; Rye (2001) Methods 24:278-288; Kenworthy (2001) Methods 24:289-296; Periasamy (2001) J. Biomed. Opt. 6:287-291; Truong & Ikura (2001) Curr. Opin. Struct. Biol. 11:573-578; Zhang, et al. (2002) Nat. Rev. Mol. Cell. Biol. 3:906-918; Sitte & Freissmuth (2003) Eur. J. Pharmacol. 479:229-236; Milligan (2004) Eur. J. Pharm. Sci. 21:397-405; Herman, et al. (2004) Methods Mol. Biol. 261:351-370; Roda, et al. (2004) Trends Biotechnol. 22:295-303; Wallrabe & Periasamy (2005) Curr. Opin. Biotechnol. 16:19-27; Milligan & Bouvier (2005) FEBS J. 272:2914-2915.

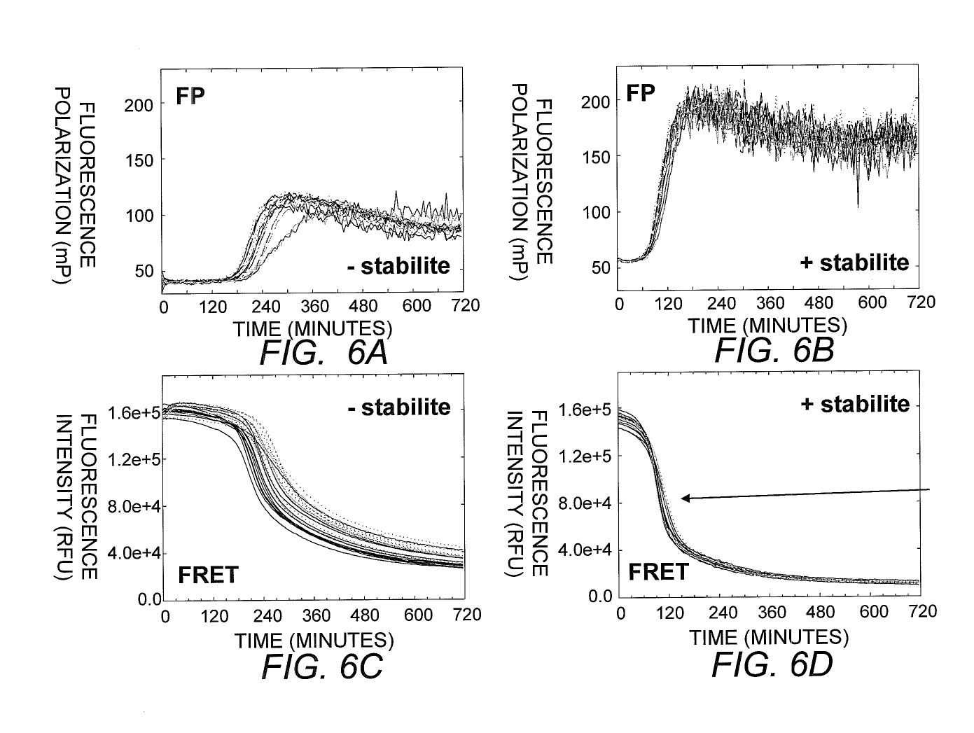

FRET methods, protocols, techniques, assays, and the like are described generally and specifically in a number of patents and patent applications, including, e.g., U.S. Pat. Nos. 6,908,769; 6,824,990; 6,762,280; 6,689,574; 6,661,909; 6,642,001; 6,639,078; 6,472,156; 6,456,734; 6,376,257; 6,348,322; 6,323,039; 6,291,201; 6,280,981; 5,914,245; 5,661,035; references in any of the foregoing; and the like. Moreover, FRET methodologies can be optimized using, e.g., stabilites such as MgCl₂ (see, e.g., FIG. 6).

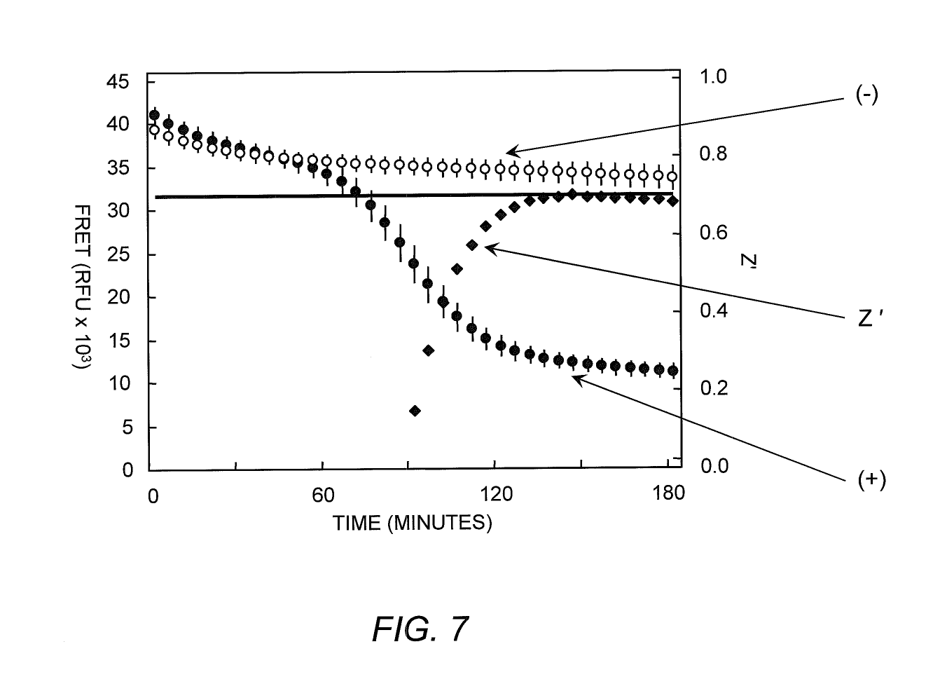

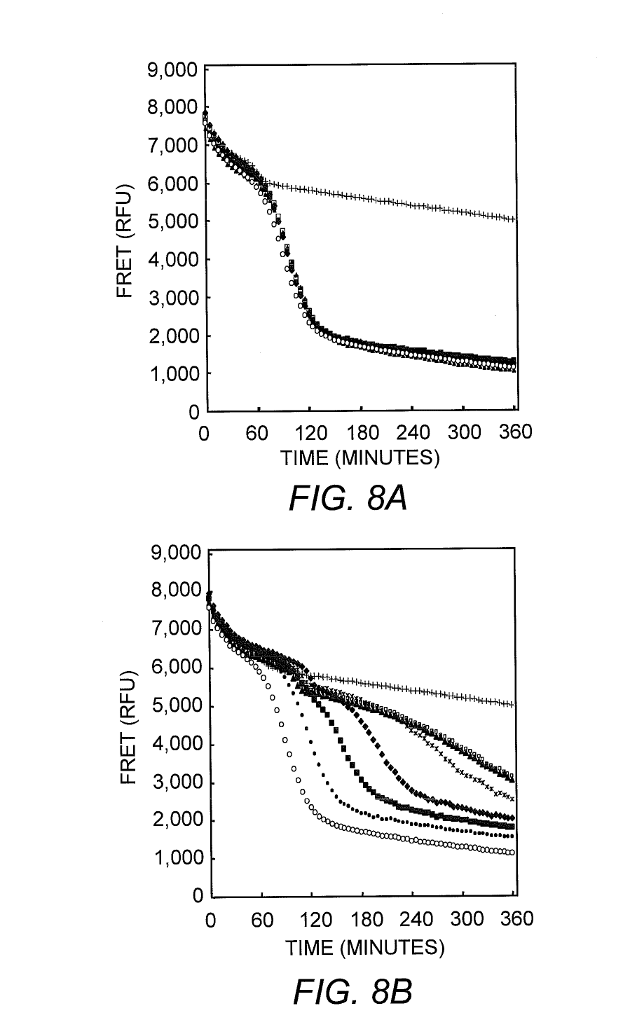

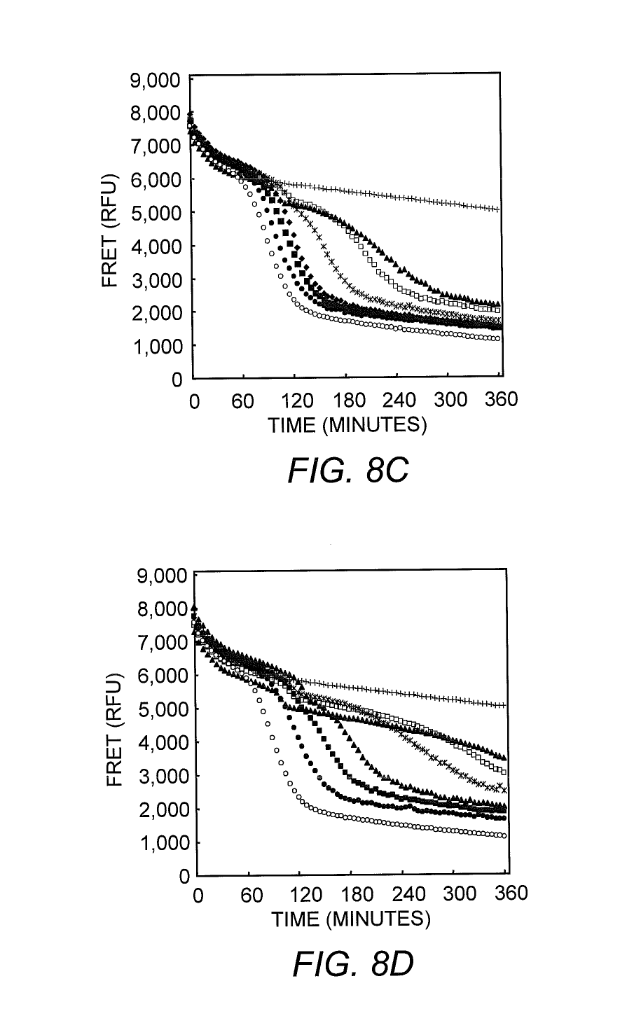

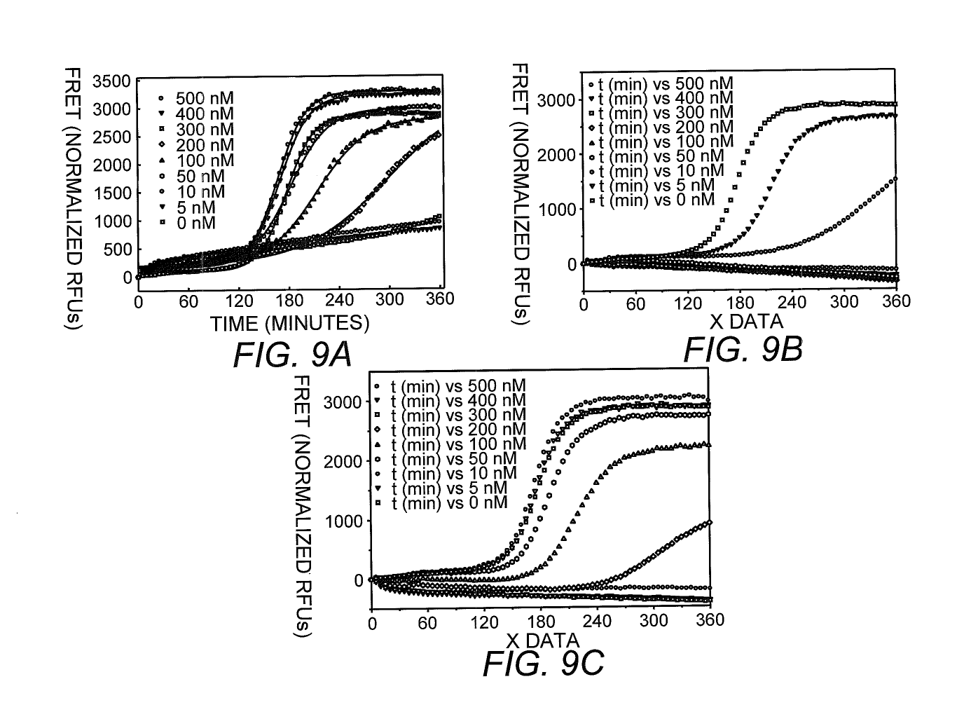

As depicted in FIGS. 7 and 8, FRET-based high throughput screening provides the identification of amyloid beta assembly inhibitors. Such assays can be used to screen small molecule libraries available from various commercial sources. Screening of such libraries, including combinatorially generated libraries (e.g., peptide libraries), is a rapid and efficient way to screen large number of related (and unrelated) compounds for activity. It is contemplated that a variety of test agents including small molecules, peptides, nucleic acids, antibodies (see, e.g., FIG. 9), etc. can be identified, assayed, analyzed, and characterized using the instant method.

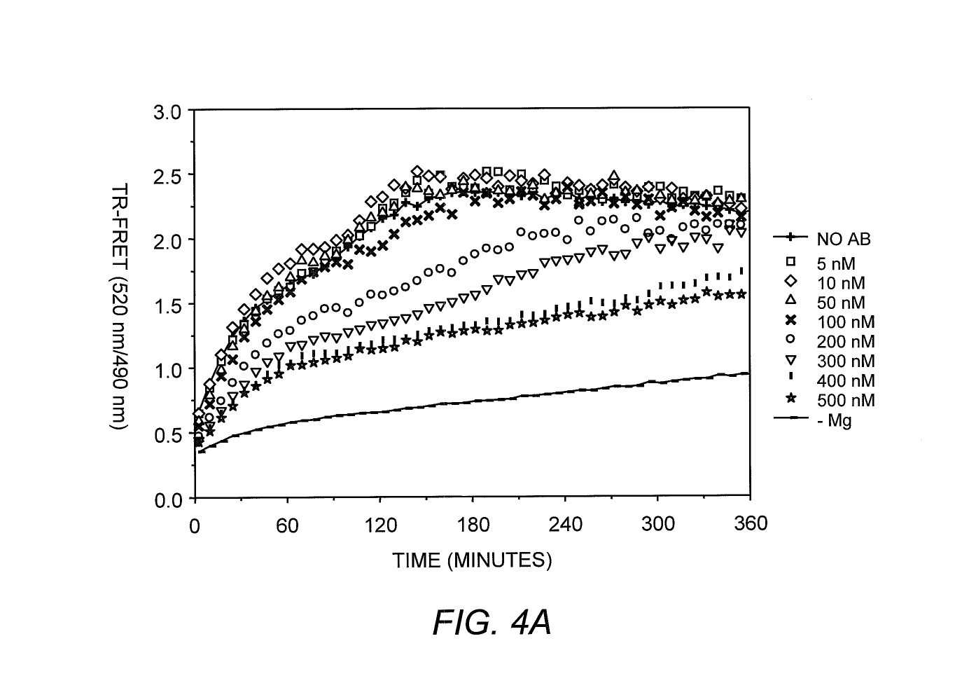

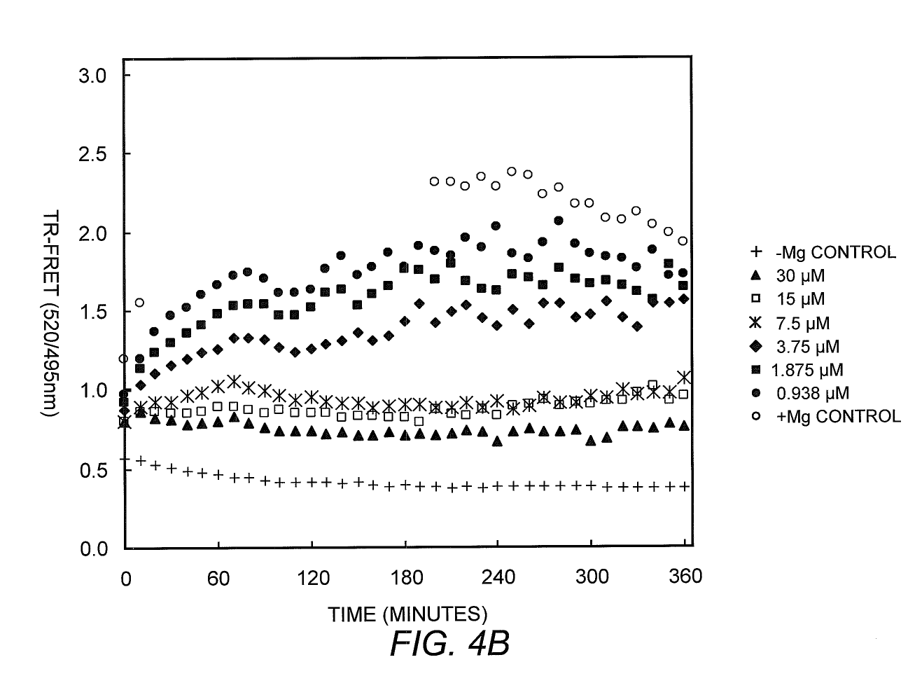

To reduce assay interference and increase data quality, particular embodiments embrace the use of a time-resolved FRET (TR-FRET) assay to detect oligomerization of soluble amyloid beta oligomers. TR-FRET generally employs a long-lifetime donor species (e.g., terbium chelate, samarium, europium, terbium, and dysprosium) and a suitable acceptor species (fluorescein or allophycocyanin), wherein the TR-FRET value is determined as a ratio of the FRET-specific signal produced by the acceptor to that of the signal produced by the donor (see FIGS. 4A and 4B). TR-FRET measurements can be carried out using any suitable technique. For example, a microscope image of donor emission can be taken with the acceptor being present. The acceptor is then bleached, such that it is incapable of accepting energy transfer and another donor emission image is acquired. A pixel based quantification using the second equation in the theory section above is then possible. An alternative way of temporarily deactivating the acceptor is based on its fluorescence saturation.

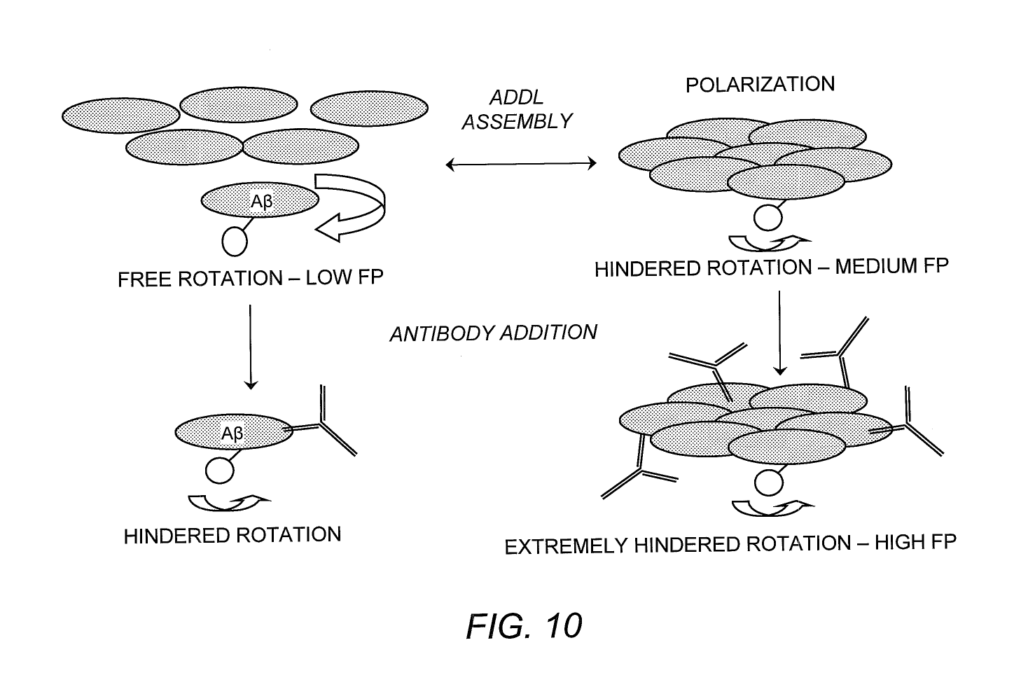

In accordance with the present invention, fluorescence polarization (FP) is also employed in the detection of soluble amyloid beta oligomerization. FP is the measurement of the polarization of fluorescent light from a sample of interest (see, e.g., FIG. 10). It is used to provide information concerning molecular size, molecular shape, conformation, electron energy transfer and molecular interactions. In this regard, FP has been used to measure, detect, identify, assay, analyze, and characterize various interactions and processes in biological systems. See, e.g., Lundblad, et al. (1996) Mol. Endocrinol. 10:607-612; Nasir & Jolley (1999) Comb. Chem. High Throughput Screen. 2:177-190; Park & Raines (2004) Methods Mol. Biol. 261:161-166; and the like.

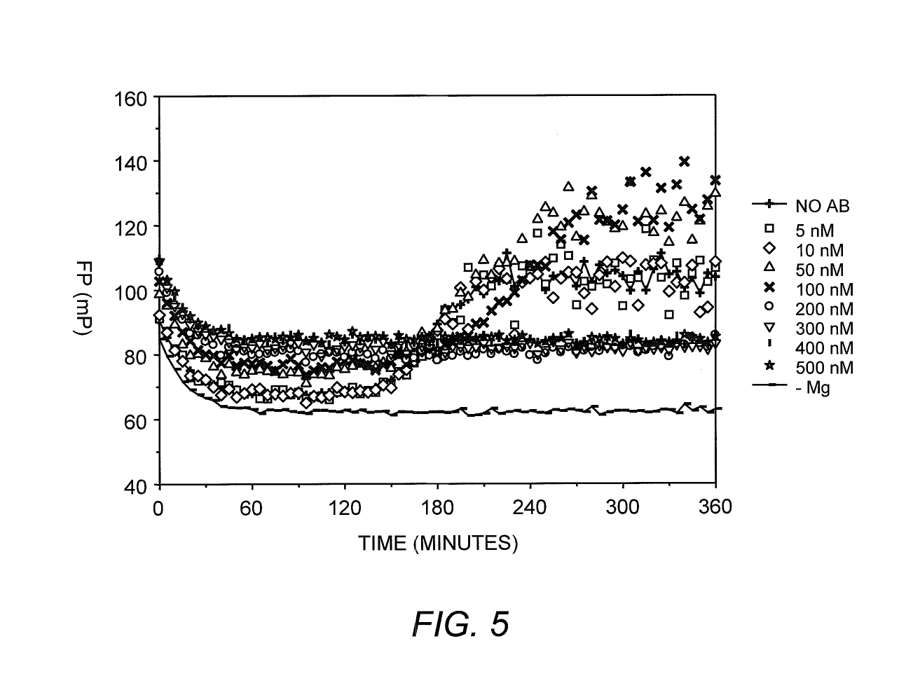

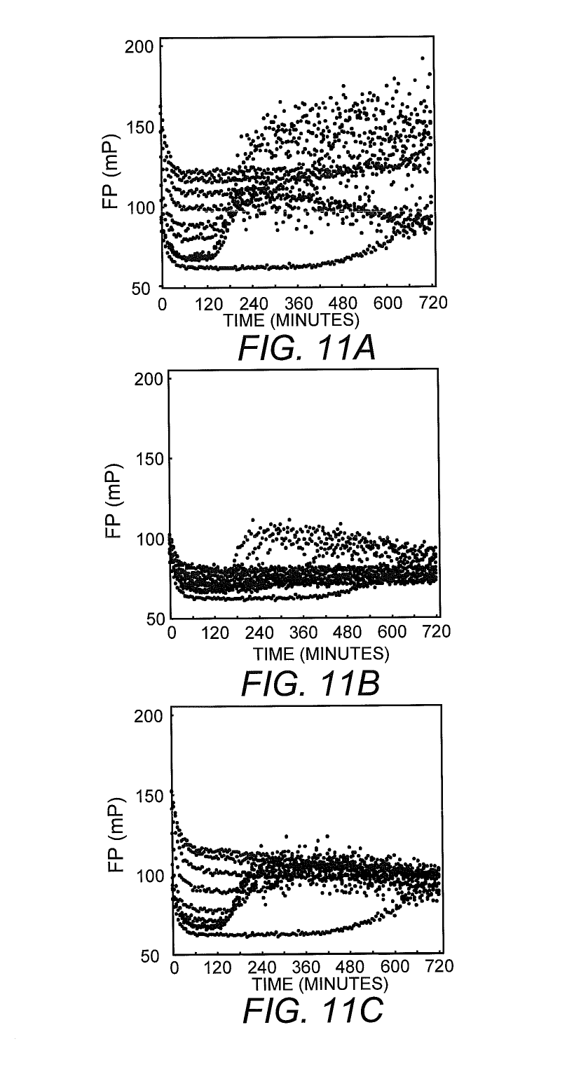

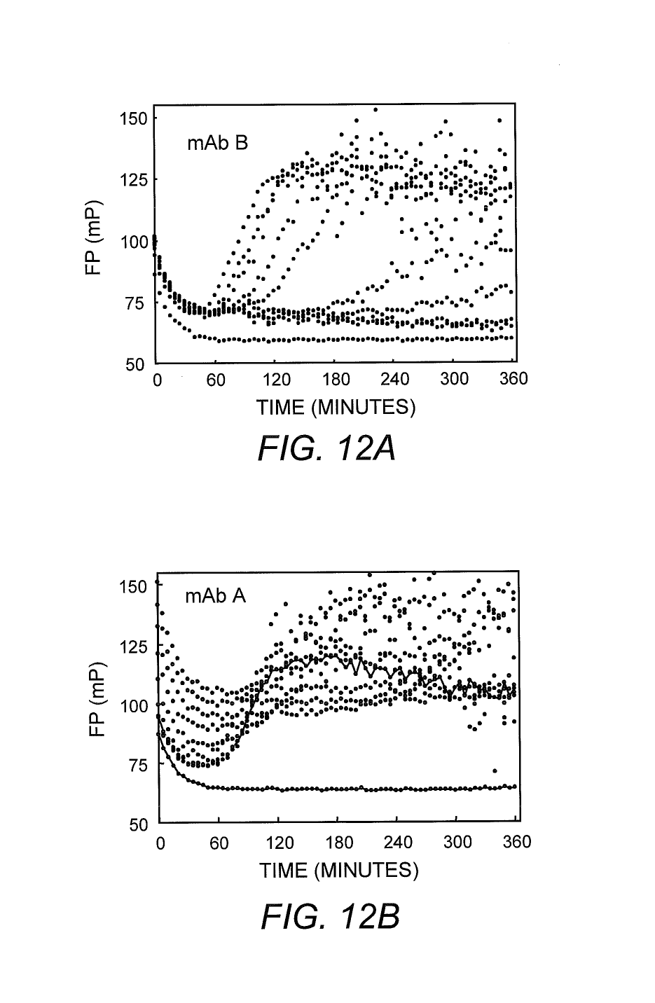

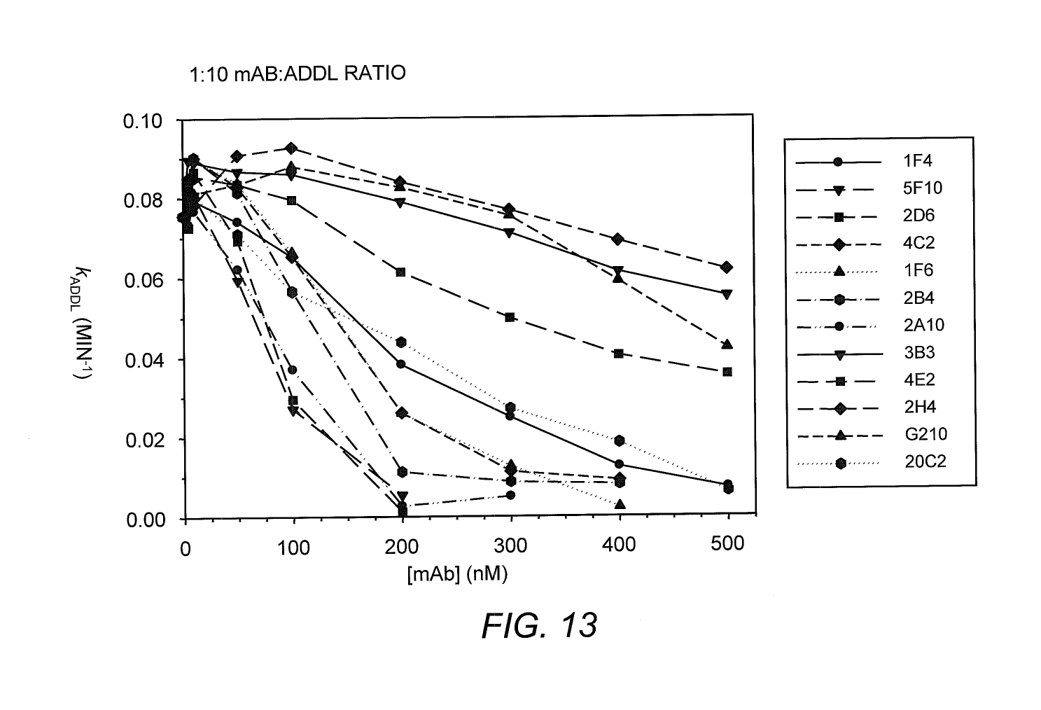

FP methods, protocols, techniques, assays are described generally and specifically in a number of patents and patent applications, including U.S. Pat. Nos. 6,794,158; 6,632,613; 6,630,295; 6,596,546; 6,569,628; 6,555,326; 6,511,815; 6,448,018; 6,432,632; 6,331,392; 6,326,142; 6,284,544; 6,207,397; 6,171,807; 6,066,505; 5,976,820; 5,804,395; 5,756,292; 5,445,935; 5,427,960; 5,407,834; 5,391,740; 5,315,015; 5,206,179; 5,070,025; 5,066,426; 4,952,691; 4,863,876; 4,751,190; 4,681,859; 4,668,640; 4,614,823; 4,585,862; 4,510,251; 4,476,229; 4,429,230; 4,420,568; 4,203,670; and the like. As depicted in FIGS. 5 and 11-12, FP-based screening is useful in the analysis of amyloid beta assembly inhibitors, such as antibodies. In particular embodiments, the method of the present invention provides the assembly kinetics of amyloid beta oligomerization (see FIG. 13).

The invention is described in greater detail by the following non-limiting examples.

### EXAMPLE 1

### Detection of Soluble Amyloid Beta Oligomers with FRET and FP

FRET and FP assays are performed in 384-well Corning Non-Binding Surface black, opaque microtiter plates, and the assay buffer consists of 50 mM MOPS-Tris (pH 8.0) with 100 mM MgCl₂. The assay volume, containing 0.2 μM FITC-Aβ(1-42) and 0.8 μM Aβ(1-42), is 50 μl and the temperature is 37° C. ADDL assembly is monitored on a Tecan GENios Pro plate reader, exciting at a wavelength of 485 nm and detecting emission at a wavelength of 515 nm. Kinetic traces are collected by recording fluorescence intensity and polarization readings every five minutes over a six-hour time course. Negative control reactions, which do not appreciably assemble into ADDLs during this time, lack MgCl₂ but contain all other buffer and peptide components. Positive control reactions contain all buffer components in the absence of added small molecule or antibody reagents. To test for ADDL binding and assembly inhibition, the antibody 6E10 was incubated with the peptide mixture at eight concentrations from 500 nM decreasing to 5 nM. To test for ADDL assembly inhibition, the small molecule was incubated with the peptide mixture at six concentrations from 30 μM decreasing to 0.9 μM. Results of such a FRET assay used to identify small molecule inhibitors of soluble amyloid beta oligomers are provided in Table 1.

TABLE 1

| Compound | Ave IC₅₀ μM |
|---|---|
| A | 1.4 |
| B | 1.6 |
| C | 1.8 |
| D | 3.2 |
| E | 5.0 |
| F | 5.1 |
| G | 5.8 |

### EXAMPLE 2

### Detection of Soluble Amyloid Beta Oligomers with TR-FRET and FP

TR-FRET assays are performed exactly as FRET and FP assays, but with different fluorophore components, concentrations and wavelength readout properties. To detect TR-FRET between FITC and Terbium, 5 nM Terbium-Streptavidin (Tb-SA) is mixed with 25 nM Biotin-Aβ(1-42), 200 nM FITC-Aβ(1-42), and 775 nM Aβ(1-42). Using the same plates, plate reader, assay buffer and assay volume as the FRET and FP assay, ADDL assembly kinetics are detected by exciting at a wavelength of 340 nm and taking the ratio of time-resolved emission at 520 nm to 490 nm using a delay time of 150 μs.

What is claimed is:

1. A method for detecting assembly of soluble amyloid beta oligomers comprising contacting a FRET-donor labeled amyloid beta subunit with a FRET-acceptor labeled amyloid beta subunit and measuring fluorescence resonance energy transfer (FRET) and fluorescence polarization (FP) of the subunits thereby detecting assembly of soluble amyloid beta oligomers.

2. The method of claim 1, wherein the contacting step is carried out in the presence of one or more inhibitors.

3. The method of claim 1, wherein the FRET is time-resolved FRET (TR-FRET).

4. A method for identifying, assaying, analyzing, and characterizing inhibitors of the assembly of soluble amyloid beta oligomers comprising contacting a FRET-donor labeled amyloid beta subunit with a FRET-acceptor labeled amyloid beta subunit in the presence of a test agent and measuring fluorescence resonance energy transfer (FRET) and fluorescence polarization (FP) of the subunits thereby identifying, assaying, analyzing, and characterizing inhibitors of the assembly of one or more soluble amyloid beta oligomers.

5. The method of claim 4, wherein the FRET is time-resolved FRET (TR-FRET).
